(19) United States
(12) Patent Application Publication
(10) Pub. No.: US 2022/0225627 A1
(43) Pub. Date: Jul. 21, 2022

(54) A NUTRITIONAL COMPOSITION FOR USE TO ENHANCE EXECUTIVE FUNCTION

(71) Applicant: SOCIETE DES PRODUITS NESTLE S.A., Vevey (CH)

(72) Inventors: Jonas Hauser, Lausanne (CH); Nora Schneider, Les Monts-de-Corsier (CH)

(21) Appl. No.: 17/434,912

(22) PCT Filed: Mar. 5, 2020
(86) PCT No.: PCT/EP2020/055767

§ 371 Date: Aug. 30, 2021

(30) Foreign Application Priority Data
Mar. 5, 2019 (EP) 19160876.9
Mar. 6, 2019 (EP) 19161023.7
Dec. 9, 2019 (EP) 19214414.5

(51) Int. Cl. A23C 9/20; A23L 33/00; A61K 35/20; A61K 31/702

(52) U.S. Cl. CPC A23C 9/206; A61K 31/702; A61K 35/20; A23L 33/40

(57) ABSTRACT

Use of a human milk oligosaccharide, or a nutritional composition comprising a human milk oligosaccharide, to enhance executive function in a subject.

### CROSS REFERENCE TO RELATED APPLICATIONS

The present application is a National Stage of International Application No. PCT/EP2020/055767, filed on Mar. 5, 2020, which claims priority to European Patent Application No. 19160876.9, filed on Mar. 5, 2019, European Patent Application No. 19161023.7, filed on Mar. 6, 2019, and European Patent Application No. 19214414.5, filed on Dec. 9, 2019, the entire contents of which are being incorporated herein by reference.

The present invention relates to the use of a human milk oligosaccharide (HMO), or a composition comprising an HMO, to enhance executive function in a subject. The invention further relates to an HMO, or a composition comprising an HMO, for use in the prevention or treatment of sub-optimal executive functioning in a subject.

### BACKGROUND OF THE INVENTION

Executive function is the ability to coordinate and integrate cognitive-perceptual processes in relation to time and space, determining how well a subject can recognise, evaluate, and make a choice among a variety of alternative options and strategies. It governs goal directed behaviour and plays a fundamental role in regulating higher-order cognitive processes such as problem solving, reasoning, flexible thinking, and decision-making. It is central to cognitive development and learning (especially of new skills), and has been strongly associated with social and intellectual/academic success/achievement.

Given the associations between executive function and social and intellectual/academic success, there is a desire to find ways to enhance it. In addition, there is also a need to find ways to treat and/or prevent sub-optimal executive functioning that can imped cognitive development and learning, and adversely affect intelligence and academic success/achievement. Depending on the severity, sub-optimal executive function can even negatively affect day to day functioning.

Sub-optimal executive functioning may be linked to a variety of cognitive conditions including Attention Deficiency Hyperactivity Disorder (ADHD), Alzheimer's disease, Obsessive Compulsive Disorder (OCD), Tourette syndrome, Post Traumatic Stress Disorder (PTSD), and vascular dementia. It is also believed to be more prevalent in subjects born preterm or small for gestational age (SGA). It is also known that executive function can decline with aging. Accordingly, there may be a particular need to enhance executive function in these patient groups.

Surprisingly the inventors have now found that consumption of an HMO may enhance executive function in a subject. This finding stems from a couple of preclinical studies, in piglets, said studies containing a combination of several tests the result of which, when considered holistically, may be considered an indication of execute function e.g. the ability of a subject to inhibit a response, to adapt a strategy, to recognise, evaluate, and to make a choice among a variety of alternative options and/or strategies.

### SUMMARY OF THE INVENTION

The invention is set out in the claims and in the detailed description included herein. The present invention provides the use of an HMO or a nutritional composition comprising an HMO to enhance executive function in a subject.

The present invention also provides an HMO or a nutritional composition comprising an HMO for use in the treatment and/or prevention of sub-optimal executive functioning in a subject.

The HMO may be a fucosylated oligosaccharide, an N-acetylated oligosaccharide and/or a sialylated oligosaccharide.

Non-limiting example of fucosylated oligosaccharide(s) include: 2′-fucosyllactose (2′FL), 3′fucosyllactose, difucosyllactose (diFL), lacto-N-fucopentaose (such as lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose (such as fucosyllacto-N-neohexaose I, fucosyllacto-N-neohexaose II), difucosyllacto-N-hexaose I, difuco-lacto-N-neohexaose, difucosyllacto-N-neohexaose I, difucosyllacto-N-neohexaose II, fucosyl-para-Lacto-N-hexaose, tri-fuco-para-Lacto-N-hexaose I and any combination thereof.

Particularly effective fucosylated oligosaccharides may be 2′-fucosyllactose (2FL) and difucosyllactose (di FL).

Non-limiting examples of N-acetylated oligosaccharide(s) include: LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

Particualrly effective N-acetylated oligosaccharides may be LNT (lacto-N-tetraose, LNnT (lacto-N-neotetraose) and combinations thereof.

Non-limiting example of sialylated oligosaccharides include: 3′-sialyllactose (3′-SL), 6′-sialyllactose (6′-SL).

In one aspect, the present invention relates to a nutritional composition comprising a sialylated oligosaccharide for improving mylenation to mature the pre-frontal cortex region thereby aiding in enhancing executive function in a human child or infant, for example a pre-term or SGA infant. The sialylated oligossacharide is 3′-SL,6′-SL or combination thereof.

Particualrly effective sialylated oligosaccharides may be 3′-sialyllactose (3′-SL), 6′-sialyllactose (6′-SL) and combinations thereof.

Accordingly, it may be particularly beneficial if the HMO is selected from the group consisting of: 2′-fucosyllactose (2′FL), diFL, LNT, LNnT, a sialylllactose and any combination of the foregoing.

The sialyllactose may be selected from the group consisting of 3′-sialyllactose (3′-SL), 6′-sialyllactose (6′-SL), and a combination thereof. It may be particularly beneficial if the sialyllactose is 6′-sialyllactose or a combination of 3′-sialyllactose (3′-SL) and 6′-sialyllactose (6′-SL).

Particularly effective combinations of HMOs may be

2′fl, diFL, LNT and LNnT,

3′-sialyllactose (3′-SL) and 6′-sialyllactose (6′-SL) and,

2′FL, diFL, LNT and LNnT, 3′-sialyllactose (3′-SL) and 6′-sialyllactose (6′-SL).

Executive function may be recognising, evaluating, and/or make choices among a variety of alternative options and/or strategies. Accordingly, an HMO, or nutritional composition comprising a HMO, may be particularly effective at enhancing a subject's ability to recognise, evaluate, and/or make a choice among a variety of alternative options and/or strategies.

The subject may be a mammal and for example may be a human or companion animal. The HMO, or composition comprising an HMO, may be particularly suited for, or particularly effective in, a human child or infant, for example a pre-term or SGA infant.

If the HMO or composition comprising an HMO is for use in the treatment and/or prevention of sub-optimal executive functioning, it may be particularly effective in a subject suffering from ADHD, Alzheimer's disease, OCD, Tourette syndrome, PTSD, or vascular dementia. It also may be particularly effective in an aging human or an infant or child that was born preterm or SGA.

A nutritional composition comprising an HMO may be an infant formula, a starter infant formula, a follow-on formula, a preterm infant formula, a fortifier, a human milk fortifier, a baby food formula, a growing-up milk, an infant cereal composition, a food product, a medical food product for clinical nutrition, a supplement, a pet food product, or supplement for pets.

The present invention also provides an HMO or a composition comprising an HMO for use in the preparation of a composition for use in the prevention and/or treatment of sub-optimal executive function in a subject

The present invention also provides a method of preventing and/or treating sub-optimal executive function in a subject, said method comprising the step of administering to said subject an HMO and/or composition comprising an HMO as disclosed herein, said method may optionally comprise the step of identifying a subject suffering from sub-optimal executive function.

The present invention also provides a method of enhancing executive function in a subject, said method comprising the step of administering to said subject an HMO and/or composition comprising an HMO as disclosed herein.

### BRIEF DESCRIPTION OF THE DRAWINGS

FIG. 1: Performance score for pigs exposed to pre-weaning diet supplemented with milk A, B, C, control milk or being reared by the sow, computed to evaluate general working memory. Different letters indicate significant differences (p<0.05)

FIG. 2: Performance score for pigs exposed to pre-weaning diet supplemented with milk A, B, C, control milk or being reared by the sow, computed to evaluate reference memory. Different letters indicate significant differences (p<0.05)

FIG. 3: Performance score for pigs exposed to pre-weaning diet supplemented with milk A, B, C, control milk or being reared by the sow, computed to evaluate working memory. Different letters indicate significant differences (p<0.05)

FIG. 4: Expression of myelin gene in the prefrontal and hippocampal brain samples of pups and adult mice that received milk without 6′SL normalized to expression level of mice receiving milk with 6′SL. Asterisks indicates significant differences to control (p<0.05).

### DETAILED DESCRIPTION OF THE INVENTION

In a first aspect of the present invention there is provided the use of an HMO, or a nutritional composition comprising an HMO, to enhance executive function in a subject.

The HMO may be a fucosylated oligosaccharide, an N-acetylated oligosaccharide, a sialylated oligosaccharide or any combination of any of the foregoing.

In an embodiment the HMO is a fucosylated oligosaccharide.

In an embodiment the HMO is an N-acetylated oligosaccharide.

In an embodiment the HMO is a sialylated oligosaccharide.

In an embodiment the HMO is a combination of one or more fucosylated oligosaccharide and one or more N-acetylated oligosaccharide.

In an embodiment the HMO is a combination of one or more fucosylated oligosaccharide and one or more N-acetylated oligosaccharide and one or more a sialylated oligosaccharide.

Non limiting example of fucosylated oligosaccharide(s) include: 2′-fucosyllactose (2′FL), 3′fucosyllactose, difucosyllactose (diFL), lacto-N-fucopentaose (such as lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose (such as fucosyllacto-N-neohexaose I, fucosyllacto-N-neohexaose II), difucosyllacto-N-hexaose I, difuco-lacto-N-neohexaose, difucosyllacto-N-neohexaose I, difucosyllacto-N-neohexaose II, fucosyl-para-Lacto-N-hexaose, tri-fuco-para-Lacto-N-hexaose I and any combination thereof.

Particualrly effective fucosylated oligosaccharides may be 2′-fucosyllactose (2FL) and difucosyllactose (di FL).

In an embodiment the fucosylated oligosaccharides is selected from the group consisting of 2′-fucosyllactose (2FL), difucosyllactose (di FL), and a combination thereof.

Non-limiting examples of N-acetylated oligosaccharide(s) include: LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

Particualrly effective N-acetylated oligosaccharides may be LNT (lacto-N-tetraose) and LNnT (lacto-N-neotetraose).

In an embodiment the N-acetylated oligosaccharides is selected from the group consisting of LNT (lacto-N-tetraose), LNnT (lacto-N-neotetraose), and a combination thereof.

Non-limiting example of sialylated oligosaccharides include: 3′-sialyllactose (3′-SL), 6′-sialyllactose (6′-SL).

In an embodiment the sialylated oligosaccharides is selected from the group consisting of: 3′-sialyllactose (3′-SL), 6′-sialyllactose (6′-SL), and a combination thereof.

In an embodiment the HMO is selected from the group consisting of 2′-fucosyllactose (2′FL), diFL, LNT, LNnT, a sialylllactose and any combination of the foregoing.

In an embodiment the HMO is a combination of 2′fl, diFL, LNT and LNnT.

In an embodiment the HMO is a combination of 3′-sialyllactose (3′-SL) and 6′-sialyllactose (6′-SL).

In an embodiment the HMO is a combination of 2′FL, diFL, LNT, LNnT, 3′-sialyllactose (3′-SL) and 6′-sialyllactose (6′-SL).

As used herein the term “3′-sialyllactose” (3′-SL, 3-SL, 3′SL, or 3SL), refers to (6R)-5-Acetamido-3,5-dideoxy-6-[(1R,2R)-1,2,3-trihydroxypropyl]-β-L-threo-hex-2-ulopyranonosyl-(2->3)-β-D-galactopyranosyl-(1->4)-D-glucopyranose (IUPAC).

As used herein the term “6′-sialyllactose” (6′-SL, 6-SL, 6′SL, or 6SL) refers to (6R)-5-Acetamido-3,5-dideoxy-6-[(1R,2R)-1,2,3-trihydroxypropyl]-β-L-threo-hex-2-ulopyranonosyl-(2->6)-β-D-galactopyranosyl-(1->4)-D-glucopyranose (IUPAC).

As used herein the term LNT (lacto-N-tetraose) refers to beta-D-galacto-hexopyranosyl-(1->3)-2-acetamido-2-deoxy-beta-D-gluco-hexopyranosyl-(1->3)-beta-D-galacto-hexopyranosyl-(1->4)-D-gluco-hexopyranose (IUPAC) As used herein the term LNnT (lacto-N-neotetraose) refers to beta-D-galacto-hexopyranosyl-(1->4)-2-acetamido-2-deoxy-beta-D-gluco-hexopyranosyl-(1->3)-beta-D-galacto-hexopyranosyl-(1->4)-D-gluco-hexopyranose (IUPAC) As used herein the term 2′-fucosyllactose (2FL) refers to (2R,3R,4R,5R)-4-[(2S,3R,4S,5R,6R)-4,5-dihydroxy-6-(hydroxymethyl)-3-[(2S,3S,4R,5S,6S)-3,4,5-trihydroxy-6-methyloxan-2-yl]oxyoxan-2-yl]oxy-2,3,5,6-tetrahydroxyhexanal (IUPAC) As used herein the term difucosyllactose (di FL) refers to (3S,4S,5S,6R)-6-(hydroxymethyl)-5-[(2S,3R,4,5R,6R)-3,4,5-trihydroxy-6-(hydroxymethyl)oxan-2-yl]oy-2,3-bis[(3S,4R,5S,6S)-3,4,5-trihydroxy-6-methyloxan-2-yl]oxane-2,3,4-triol (IUPAC) The N-acetylated oligosaccharide(s) e.g. LNnT and/or LNT, may be synthesised chemically by enzymatic transfer of saccharide units from donor moieties to acceptor moieties using glycosyltransferases as described for example in U.S. Pat. No. 5,288,637 and WO 96/10086.

Alternatively, LNT and LNnT may be prepared by chemical conversion of Keto-hexoses (e.g. fructose) either free or bound to an oligosaccharide (e.g. lactulose) into N-acetylhexosamine or an N-acetylhexosamine-containing oligosaccharide as described in Wrodnigg, T. M.; Stutz, A. E. (1999) Angew. Chem. Int. Ed. 38:827-828. N-acetyl-lactosamine produced in this way may then be transferred to lactose as the acceptor moiety.

The sialylated oligosaccharide(s) e.g. 3′-sialyllactose (3′-SL), and/or 6′-sialyllactose (6′-SL), may be isolated by chromatographic or filtration technology from a natural source such as animal milks.

Alternatively, they may be produced by biotechnological means using specific sialyltransferases or sialidases, neuraminidases, either by an enzyme based fermentation technology (recombinant or natural enzymes), by chemical synthesis or by a microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP), from DP=1 onwards. Alternatively, sialyllactoses may be produced by chemical synthesis from lactose and free N′-acetylneuraminic acid (sialic acid). Sialyllactoses are also commercially available for example from Kyowa Hakko Kogyo, Japan, or from GeneChem, Republic of Korea.

The fucosylated oligosaccharide(s) e.g. 2′FL and/or diFL may be isolated by chromatography or filtration technology from a natural source such as animal milks. Alternatively, it may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Fucosylated oligosaccharide formation can be initiated by acceptor substrates starting from any degree of polymerization (DP), from DP=1 onwards. Alternatively, fucosylated oligosaccharides may be produced by chemical synthesis from lactose and free fucose. Fucosylated oligosaccharides are also available for example from Kyowa, Hakko, Kogyo of Japan.

The term “executive function” as used herein refers to the ability to recognise, evaluate, and make a choice among a variety of alternative options and strategies. The term encompasses goal directed behaviour, planning, and/or cognitive flexibility.

The term “subject as used herein” refers to a mammal and may for example be a human or an animal such as a companion animal e.g. a cat or a dog.

In an embodiment of the invention the subject is a human or a companion animal e.g. a cat or a dog. A human may be an infant, a young child, a child, a teenager or an adult including an aging adult.

An aging adult may be a human of 50 years of age or older, for example 60 years of age or older, 70 years of age or older, 80 years of age or older, 90 years of age or older. A human infant is a human of 12 months or younger. A “young child” is a human between one and seven years of age for example between 1 and three years of age. A “child” may be a young child.

An infant may be a preterm infant, a small for gestational age (SGA) infant and/or an infant with a low birth weight (LBW).

The term “preterm” or “premature” means an infant or young child who was not born at term. Generally it refers to an infant or young child born prior 36 weeks of gestation.

By the expression “small for gestational age” or “SGA” it is referred to an infant or young child who is smaller in size than normal for their gestational age at birth, most commonly defined as a weight below the 10th percentile for the gestational age. In some embodiments, SGA may be associated with Intrauterine growth restriction (IUGR), which refers to a condition in which a foetus is unable to achieve its potential size.

The expression “low birth weight” is to be understood as any body weight under 2500 g at birth.

It therefore encompasses:
an infant or young child who has/had a body weight from 1800 to 2500 g at birth (usually called “low birth weight” or LBW)
an infant or young child who has/had a body weight from 1000 to 1800 g at birth (called “very low birth weight” or VLBW)
an infant or young child who has/had a body weight under 1000 g at birth (called “extremely low birth weight” or ELBW)

Infants or young children with low birth weight may or may not be preterm, and similarly, infants or young children who were small for gestational age may or may not be preterm.

HMOs are compounds found in human breast milk (human milk oligosaccharides), accordingly, it may be particularly beneficial if an HMO, or composition comprising an HMO, is administered to an infant or child, and in particular to an infant or child fed infant formula or growing up milk. Whilst breast-feeding is recommended for all infants, in some cases breast-feeding is insufficient or not possible for medical reasons. In these situations infant formula or growing up milks are a lifeline as they can be used as an alternative to mother's milk.

Accordingly, in an embodiment, the subject is a human infant or young child, and in a more specific embodiment still the subject is a human infant or child fed infant formula or growing up milk.

The subject may be a healthy subject not suffering from sub-optimal executive function.

Executive function may be measured by methods well known to the skilled person.

for example by assessing, possibly holistically assessing, different cognitive skills known to make up executive function e.g. inhibition, working memory, cognitive flexibility, pattern recognition tests and the like.

In children for example Dimensional Change Card Sort (DCCS) task, in adults for examples Wisconsin Card Sorting Task. Participants are shown target cards and asked to sort a series of bivalent test cards according to one dimension (e.g., color). During a post-switch phase, they are told to sort the same types of test cards according to the other dimension (e.g., shape).

A subject not suffering from sub-optimal executive functioning would have test scores within ranges deemed normal (non pathological) for example for the type and age of the subject.

The HMO or nutritional composition comprising an HMO may be administered to a lactating mammal and thereby to an infant via breastfeeding. Without wishing to be bound by theory, the inventors believe that HMOs or metabolites thereof may be transferred to the infant via breastmilk.

The HMO or nutritional composition comprising an HMO may also be administered to a pregnant mammal or a mammal trying to get pregnant (pre-pregnancy) and thereby to an infant in-utero. Without wishing to be bound by theory, the inventors believe that an HMO or metabolites thereof may be transferred to the infant in-utero.

Accordingly, in another embodiment of the invention administration of an HMO, or composition comprising an HMO, to the infant is postnatally via breastfeeding.

The composition comprising an HMO may be any type of composition suitable for consumption by a subject.

In an embodiment of the invention the composition is selected from the group consisting of an infant formula, a starter infant formula, a follow-on formula, a preterm infant formula, a fortifier, a human milk fortifier, a baby food formula, a growing-up milk, an infant cereal composition, a food product, a medical food product for clinical nutrition, a supplement, a pet food product, or a supplement for pets.

In a more specific embodiment of the invention the composition comprising an HMO is an infant formula, a human milk fortifier, or a supplement.

A medical food product is specially formulated and intended for the dietary management of diseases or medical conditions (e.g., to prevent or treat undesirable medical conditions). A medical food product can provide clinical nutrition, for example fulfilling special nutritional needs of patients with a medical condition or other persons with specific nutritional needs. A medical food product can be in the form of a complete meal, part of a meal, as a food additive, or a powder for dissolution.

A food product, medical food or nutritional composition can be in any oral nutritional form, e.g. as a health drink, as a ready-made drink, optionally as a soft drink, including juices, milk-shake, yogurt drink, smoothie or soy-based drink, in a bar, or dispersed in foods of any sort, such as baked products, cereal bars, dairy bars, snack-foods, soups, breakfast cereals, muesli, candies, tabs, cookies, biscuits, crackers (such as a rice crackers), and dairy products.

A supplement may for example be in the form of tablets, capsules, pastilles or a liquid. The supplement can be added in a product acceptable to the consumer as an ingestible carrier or support. Non-limiting examples of such carriers or supports are a pharmaceutical, a food composition. Non-limiting examples for food compositions are milks, yogurts, curds, cheeses, fermented milks, milk-based fermented products, fermented cereal based products, milk-based powders, human milks, preterm formulas, infant formulas, oral supplements, and tube feedings.

The term “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

Generally a “starter infant formula” is intended for infants from birth as breast-milk substitute.

A “follow-up formula” or “follow-on formula” is given from the 6th month onwards. It constitutes the principal liquid element in the progressively diversified diet of this category of person.

The term “preterm infant formula” as used herein means an infant formula intended for a preterm infant.

The term “milk fortifier” as used herein refers to liquid or solid nutritional compositions suitable for mixing with breast milk (which is human milk for a human milk fortifier) or infant formula. It is used to increase the calories, protein, minerals and vitamins in breast milk fed to preterm infants or infants with a low birth weight. The term “breast milk” is to be understood as the mother's milk or the colostrum of the mother or a donor's milk or the colostrum of a donor's milk.

The term “baby food formula” as used herein means a foodstuff intended for particular nutritional use by infants or children such as young children, during the first years of life.

The term “growing-up milk” (or GUM) as used herein refers to a milk formula product given from one year onwards. It is generally a diary-based beverage adapted for the specific nutritional needs of young children.

The term “infant cereal composition” as used herein refers to a foodstuff intended for particular nutritional use by infants or children such as young children, during the first years of life.

In addition to an HMO, the compositions of the invention can also comprise any other ingredients or excipients known to be employed in the type of composition in question e.g. infant formula.

Non-limiting examples of such ingredients include: proteins, amino acids, carbohydrates, oligosaccharides (other than HMOs), lipids, prebiotics or probiotics, nucleotides, nucleosides, other vitamins, minerals and other micronutrients.

If the composition is a composition for an infant or young child, the composition may for example comprise a protein source, a lipid source and a carbohydrate source. For example such a composition may comprise protein in the range of about 2 to 6 g/100 kcal, lipids in the range of about 1.5 to 3 g/100 kcal and/or carbohydrates in the range of about 1.7 to 12 g/100 kcal. If said composition is liquid, its energy density may be between 60 and 75 kcal/100 ml. If said composition is solid, its energy density may be between 60 and 75 kcal/100 g.

Non-limiting examples of proteins include: casein, alpha-lactalbumin, whey, beta lactoglobulin, soy protein, rice protein, corn protein, oat protein, barley protein, wheat protein, rye protein, pea protein, egg protein, sunflower seed protein, potato protein, fish protein, meat protein, lactoferrin, serum albumin, immunoglobins, and combinations thereof.

Non-limiting examples of amino acids include leucine, threonine, tyrosine, Isoleucine, arginine, alanine, histidine, isoleucine, proline, valine, cysteine, glutamine, glutamic acid, glycine, L-serine, arginine, lysine, methionine, phenylalanine, tryptophane, asparagine, aspartic acid, and combinations thereof.

Non-limiting examples of carbohydrates include lactose, saccharose, maltodexirin, starch, and combinations thereof.

Nonlimiting examples of lipids include: palm olein, high oleic sunflower oil, high oleic safflower oil, canola oil, fish oil, coconut oil, bovine milk fat, and combinations thereof.

It may be particularly beneficial if the composition comprises fat in an amount of 25 to 30 g/100 g dry weight of the composition.

Non-limiting examples of essential fatty acids include: linoleic acid (LA), α-linolenic acid (ALA). The compositions of the invention may further contain gangliosides. Non limiting examples of gangliosides include: monosialoganglioside-3 (GM3) and disialogangliosides 3 (GD3), and combinations thereof.

Non limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

Further examples of oligosaccharide are described in Wrodnigg, T. M.; Stutz, A. E. (1999) Angew. Chem. Int. Ed. 38:827-828 and in WO 2012/069416 which is incorporated herein by reference.

Non-limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM 1-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1.3724), Enterococcus faecium SF 68 (NCC2768; NCIMB10415), and combinations thereof.

Non-limiting examples of Nucleotides include: cytidine monophosphate (CMP), uridine monophosphate (UMP), adenosine monophosphate (AMP), guanosine monophosphate (GMP), and combinations thereof.

The composition comprising an HMO, can further comprise at least one non-digestible oligosaccharide (e.g. prebiotics) in addition to the HMO. Examples of such prebiotics include certain oligosaccharides, such as fructooligosaccharides (FOS), galactooligosaccharides (GOS), fucosylated oligosaccharides (such as 2′-fucosyllactose, 3′fucosyllactose, difucosyllactose, lacto-N-fucopentaose I, lacto-N-fucopentaose II, lacto-N-fucopentaose III, lacto-N-fucopentaose V, lacto-N-fucohexaose, lacto-N-difucohexaose I, fucosyllacto-N-hexaose, fucosyllacto-N-neohexaose I, fucosyllacto-N-neohexaose II, difucosyllacto-N-hexaose I, difucosyllacto-N-neohexaose I, difucosyllacto-N-neohexaose II, fucosyl-para-Lacto-N-hexaose, and any combination thereof), N-acetylated oligosaccharides (such as lacto-N-tetraose (LNT), N-neotetraose (LNnT) and any combination thereof). They could be usually in an amount between 0.3 and 10% by weight of composition.

Other suitable and desirable ingredients of compositions that may be employed in the composition of the invention may be described in guidelines issued by the Codex Alimentarius with respect to the type of composition in question e.g. Infant formula, HM fortifier, follow on formula, or food stuffs intended for consumption by infants e.g. infant cereals.

The composition comprising an HMO, for example an infant formula, may be prepared in any suitable manner. For example, an infant formula may be prepared by blending together a protein source, a carbohydrate source, and a fat source in appropriate proportions. If used, emulsifiers may be included in the blend. An HMO may be added at this point, any vitamins and any minerals may also be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger. The liquid mixture may then be cooled to about 60° C. to about 85° C.; for example by flash cooling. The liquid mixture may then be homogenised; for example in two stages at about 7 MPa to about 40 MPa in the first stage and about 2 MPa to about 14 MPa in the second stage. The homogenised mixture may then be further cooled to add any heat sensitive components; such as vitamins and minerals. The pH and solids content of the homogenised mixture is conveniently standardised at this point. The homogenised mixture is transferred to a suitable drying apparatus such as a spray drier or freeze drier and converted to powder. The powder should have a moisture content of less than about 5% by weight. If it is desired to add probiotic(s), they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the powdered infant formula by dry mixing.

The composition comprising an HMO may comprise an HMO in any effective amount. It is well within the purview of the skilled person to identify an effective amount based on the nature, purpose, the target subject and the dosage of the composition e.g. how many times per day the composition is to be ingested by the subject. Typically an effective amount will depend on age, size and health status of the subject, on the subject's lifestyle and on the dosage of the composition.

An effective amount may be any amount that enhances executive function in a subject.

Enhancements in executive function may be measured by well-known tests as detailed hereinabove.

The enhancement of executive function may only be detectable after more than 6 months, 1 year, for example more than 5 years, more than 10 years, more than 20 years.

It is well within the purview of the skilled person to determine an effective dose based upon the information herein and the knowledge in the field.

For an infant formula or growing up milk, the skilled person may base the amount of an HMO e.g. 2′FL, diFL, LNT, LNnT, 3SL and/or 6SL on the amounts found in human breast milk produced for an infant or child of the same age, in particular by a nutritionally replete mother. Such amounts may fall within the following ranges in human breast milk: diFL:100-500 mg/L, LNT: 50-300 mg/L, LNnT: 200-2000 mg/L, 2′FL: 500-3000 mg/L, 3′SL: 100-400 mg/L, 6′SL: 50-750 mg/L. However, they may be outside depending on for example bioavailability of said HMOs from infant formula in comparison to human breastmilk.

As a guide, for an infant formula or growing up milk, the fucosylated oligosaccharide(s) e.g. 2′FL and/or diFL, may be present in the nutritional composition according to the present invention in a total amount of 0.75-1.65 g/L of the composition, for example in a total amount of 0.8-1.5 g/L of the composition for example 0.85-1.3 g/L, 0.9-1.25 g/L, 0.9-1.1 g/L, 1-1.25 g/L, 1.05-1.25 g/L of the composition (the concentration may refer to the concentration after the composition has been reconstituted e.g. with water).

As a guide, for an infant formula or growing up milk, the N-acetylated oligosaccharide(s) e.g. LNT and/or LNnT) may be present in the nutritional composition according to the present invention in a total amount of 0.45-0.9 g/L of the composition, for example in a total amount of 0.5 g/L of the composition, for example 0.63 g/L of the composition.

As a guide, for an infant formula or growing up milk, the sialylated oligosaccharide (s) e.g. sialyllactose (3′-sialyllactose (3′-SL) and/or 6′-sialyllactose (6′-SL)) may be present in the nutritional composition according to the invention in a concentration of from 50 mg to 5000 mg/L for example from 50 mg to 2500 mg/L for example from 60 mg to 2000 mg per L, from 80 mg to 1000 mg per L of the nutritional composition. In a particular embodiment, the composition comprises 2090 mg of total sialyllactose per L of composition. In another particular embodiment the composition comprises from 87.5 mg to 735 mg of total sialyllactose per L of the nutritional composition.

If the composition comprising an HMO comprises 3′-Sialyllactose (3′-SL) and 6′-Sialyllactose (6′-SL), it may be particularly beneficial if said 3′-Sialyllactose (3′-SL) and 6′-Sialyllactose (6′-SL) are comprised in said nutritional composition in a weight ratio between 10:1 and 1:10, such as between 10:1 and 2:1, between 8:1 and 3:1, between 6:1 and 3:1, between 5:1 and 3:1, between 5:1 and 4:1, or between 4.7:1 and 4.1:1.

As would be evident to the skilled person, the HMO or composition comprising an HMO as disclosed herein for use to enhance executive function, may also be used in the prevention and/or treatment of sub-optimal executive function in a subject.

Accordingly, in another aspect of the present invention there is provided an HMO or composition comprising an HMO, as disclosed herein, for use in the prevention and/or treatment of sub-optimal executive functioning.

In an embodiment the subject may be a subject suffering from sub-optimal executive function and therefor in need of an enhancement in executive functioning.

A person suffering from sub-optimal executive function may be a subject that does not have test scores (in standard test used to assess executive function) within ranges deemed normal (non pathological) e.g. for the type and age of the subject. It is well within the purview of the person skilled in the art to determine when a subject is suffering from sub-optimal executive functioning.

Sub-optimal executive functioning may be linked to a variety of cognitive conditions including Attention Deficiency Hyperactivity Disorder (ADHD), Alzheimer's disease, and vascular dementia.

It is also believed to be more prevalent in subjects born preterm or small for gestational age (SGA) and, it is known that executive function can decline with aging. There may therefore be a particular need to treat and/or prevent sub-optimal executive functioning in these patient groups.

Accordingly, in a more particular embodiment the subject in need of an enhancement in executive function is a subject suffering from ADHD, Alzheimer's disease, or vascular dementia, or is an aging adult, an infant born preterm or small for gestational age (SGA).

In another aspect of the preset invention there is provided the use of an HMO or composition comprising an HMO, as disclosed herein, for use in the preparation of a composition for use in the prevention and/or treatment of sub-optimal executive functioning.

In another aspect of the present invention there is provided a method of preventing and/or treating sub-optimal executive function in a subject, said method comprising the step of administering to said subject an HMO or composition comprising an HMO, as disclosed herein. Said method may also optionally comprise the step of identifying a subject suffering from sub-optimal executive functioning.

In another aspect of the present invention there is provided a method of enhancing executive function in a subject, said method comprising the step of administering to said subject an HMO or composition comprising an HMO as disclosed herein, said method may optionally comprise the step of identifying a subject suffering from sub-optimal executive function.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

As used in this disclosure and the appended claims, the singular forms “a,” “an” and “the” include plural referents unless the context clearly dictates otherwise. Thus, for example, reference to “an ingredient” or “the ingredient” includes two or more ingredients. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. A range that is “between” two values includes those two values. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the composition unless expressed otherwise. When reference is made to the pH, values correspond to pH measured at 25° C. with standard equipment.

The relative term “enhance” and “decrease” refer to the effects of an HMO or a composition comprising an HMO as disclosed herein on executive function (for example the effect on different cognitive skills known to makeup executive function e.g. attention or impulsivity, working memory, cognitive flexibility. This may need to be considered holistically) in a subject in comparison to a subject that is not administered an HMO or composition comprising an HMO. It is well within the purview of the skilled person to assess an improvements, increases or enhancements. The enhancement of executive function (different cognitive skills known to make up executive function e.g. cognitive flexibility, working memory, attention and/or decrease in impulsivity) may only be detectable after more than 1 year, for example more than 5 years, more than 10 years, more than 20 years.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Moreover, the description of some steps as “optional” does not imply that the other steps which are not explicitly described as optional are necessarily required. Where the text refers simply to a composition, this may be a nutritional composition.

There now follows a series of non-limiting examples that serve to illustrate the invention.

### EXAMPLES

### Example 1

### Methods—Behavioural Study

53 female piglets (Gottingen minipigs, Ellegaard, Danemark) were subjected to the holeboard tests after weaning. At 1 week of age, formula-fed (N=45) piglets were separated from the sows, and housed in mixed groups of 2 piglets in pens (2.5 m×1 m) enriched with shredded straw as bedding, and equipped with a squeeze ball and a dog bed. In addition, 8 piglets were cross-fostered and kept with 3 sows until 10 weeks of age (natural suckling piglets). After being weaned from the maternal milk, at 10 weeks of age, the natural suckling piglets were housed in the same conditions than the formula-fed piglets. Two metallic chains were also added to the pens as additional enrichment material.

Treatments.

The milk intervention was from 1 to 10 weeks of age. At 1 week of age, the formula-fed piglets were randomly allocated to 1 of 4 milk formulas, enriched with different prebiotic mix (milk A: 3′SL and 6′SL, milk B: 2′FL+diFL+LNT+LNnT, milk C: 2′FL+diFL+LNT+LNnT+3′SL+6′SL) or with no prebiotics (control milk). The natural suckling piglets were fed the maternal milk during the whole period of nutritional intervention. After being weaned from the milk, at 10 weeks of age piglets were fed a high-energy obesogenic diet. This experimental setting resulted in 5 treatment groups: milk A (N=12), milk B (N=12), milk C (N=10), control milk (N=11) and natural suckling (N=8).

Both pigs of each pen were individually tested in a spatial holeboard task to assess their spatial cognitive (memory and learning) performance. The holeboard arena (3 m×3 m) had black, wooden, 80-cm-high walls and 4 entrances with guillotine doors. In the arena, 16 grey metallic buckets (012 cm-H12 cm) were screwed to the floor in a 4×4 matrix, 4 of which were baited with small pieces of apple (^{˜}12×12×20 mm). Piglets inside the arena were able to see the walls of the room and the ceiling with the ventilation duct and the light tubes. Piglets were deprived from feed overnight during the whole period of holeboard testing.

From approximately 16.5 to 19 weeks of age, piglets were individually subjected to 2 massed trials (i.e. performed a few minutes apart) per day on 12 consecutive working days, i.e. 24 acquisition trials. Different entrances were used daily, with 2 different entrances per day of test (i.e. 1 entrance per trial). The trial started when the piglet had its 4 legs in the holeboard arena and ended when the piglet found all 4 rewards or after 180 s. Every time the piglet visited a baited bucket for the first time, a clicker sound was produced to facilitate learning. If the piglet completed the task (i.e. found the 4 rewards in fewer than 180 s), the exit (south) guillotine door was opened, the piglet received half of a white grape. If the piglet did not complete the task within the 180 s, a police siren sound was produced; the piglet did not receive a reward. After the 2 piglets per pen had been tested twice, all the pen mates were guided back into their home pen and their morning milk ration was distributed.

After the acquisition phase was completed, piglets were individually subjected to 16 reversal trials, with 2 massed trials per day on 8 consecutive working days. The procedure was the same procedure as in the acquisition phase, but piglets were assigned to a different configuration of baited buckets.

The following parameters were scored live using The Observer XT 10 (Noldus Information Technology, Wageningen, The Netherlands): all visits and revisits to all buckets, latencies to all bucket visits, trial duration, total number of defecations, urinations and escape attempts during the trial. From the parameters recorded during the test, variables were calculated a posteriori according to van der Staay et al. (2012. Neurosci and biobehav rev 36, 379-403) (Table 1).

TABLE 1
Parameters calculated a posteriori in the holeboard task

| Description | Calculation |
|---|---|
| Working memory (WM) scores | Number of rewarded visits/ |
| (ability of pigs to avoid revisits to | number of visits and revisits |
| baited buckets during a trial (short- | to the set of baited buckets |
| term memory)) | |
| Reference memory (RM) scores | Number of visits and revisits |
| (ability of pigs to discriminate between | to the set of baited buckets/ |
| baited and unbaited buckets (long-term | number of visits and revisits |
| memory)) | to all buckets |
| General WM scores (ability of pigs to | Number of different buckets |
| avoid revisits to buckets already visited | visited/number of visits and |
| during a trial (short-term memory)) | revisits to all buckets |

During the acquisition phase, all pigs exhibited an increase of performance visible by WM, RM and general WM score increasing linearly overtime, and the number of WM, RM and general WM errors decreasing linearly over time. The milk treatment had no effects on the cognitive performance of piglets after weaning.

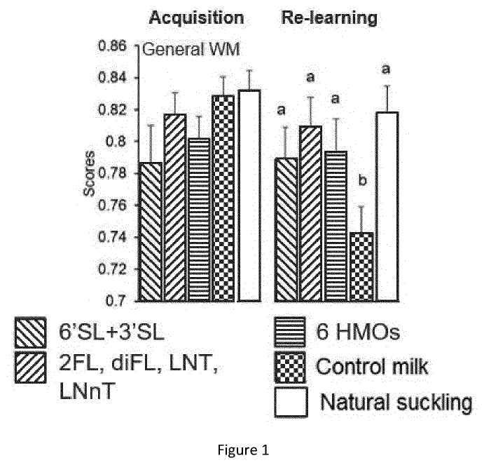

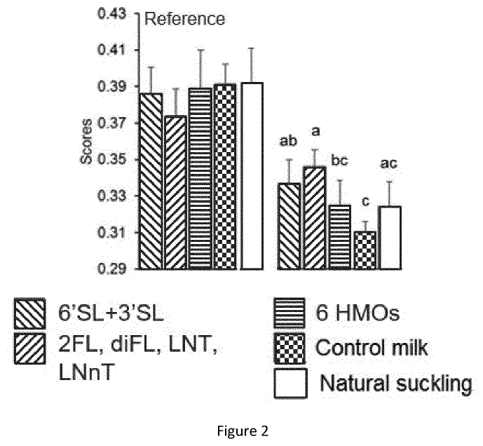

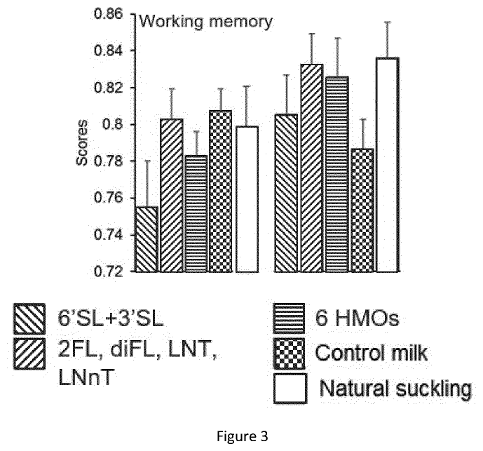

During the reversal phase, all pigs exhibited an increase of performance visible by WM, RM and general WM score increasing linearly over time, and the number of WM, RM and general WM errors decreasing linearly over time. This increase of RM, WM and general WM performance was decreased (significantly for general WM and RM) in the control milk replacer group compared to natural suckling piglets and, surprisingly, this deficit was restored by the presence of HMOs in the milk replacer to similar levels as observed in the natural suckling group (see FIGS. 1-3).

### Example 2

An example of the composition of a nutritional composition (e.g. an infant formula) according to the present invention is given in the below table 2. This composition is given by way of illustration only.

TABLE 2
an example of the composition of a nutritional composition
(e.g. an infant formula) according to the present invention

| Nutrients | | per litre |
|---|---|---|
| Energy (kcal) | | 670 |
| Protein (g) | | 12.3 |
| Fat (g) | | 35.7 |
| Linoleic acid (g) | | 5.3 |
| α-Linolenic acid (mg) | | 675 |
| Lactose (g) | | 74.7 |
| Minerals (g) | | 2.5 |
| Na (mg) | | 150 |
| K (mg) | | 590 |
| Cl (mg) | | 430 |
| Ca (mg) | | 410 |
| P (mg) | | 210 |
| Mg (mg) | | 50 |
| Mn (μg) | | 50 |
| Se (μg) | | 13 |
| Vitamin A (μg RE) | | 700 |
| Vitamin D (μg) | | 10 |
| Vitamin E (mg TE) | | 5.4 |
| Vitamin K1 (μg) | | 54 |
| Vitamin C (mg) | | 67 |
| Vitamin B1 (mg) | | 0.47 |
| Vitamin B2 (mg) | | 1.0 |
| Niacin (mg) | | 6.7 |
| Vitamin B6 (mg) | | 0.50 |
| Folic acid (μg) | | 60 |
| Pantothenic acid (mg) | | 3 |
| Vitamin B12 (μg) | | 2 |
| Biotin (μg) | | 15 |
| Choline (mg) | | 67 |
| Fe (mg) | | 8 |
| I (μg) | | 100 |
| Cu (mg) | | 0.4 |
| Zn (mg) | | 5 |
| Oligosaccharides | 2FL (g) | 1 |
| (HMOs) | LNnT (g) | 0.45 |
| | LNT(g) | 0.45 |
| | diFL (g) | 0.5 |
| | 3′SL (g) | 0.1 |
| | 6′SL (g) | 0.9 |

### Example 3

### Animals and Rearing Conditions

Adult wild-type (WT) B6.129 and heterozygous (HZ) B6.129-St6gal1^{tm2Jxm} breeding pairs (four males and four females and three males and four females, respectively) were purchased from a commercial breeder (The Jackson Laboratory). Upon arrival, same-sex mice were housed in same-sex groups of 2-3 in type-1 polycarbonate cages (33.0×13.0×14.0 cm) equipped with sawdust bedding, an enrichment bag (Mucedola, Settimo Milanese, Italy), metal top and ad libitum water and food pellets (Mucedola, Settimo Milanese, Italy). Mice were maintained on a reversed 12-h-light-dark cycle (light on at 7:00 PM) in an air-conditioned room (temperature 21±1C and relative humidity 60±10%). Two weeks after arrival, breeding triads (one male, two females) were formed. After two weeks of mating, male mice were removed and females were housed individually in standard type-1 cages. Females were checked daily for delivery and the day in which they gave birth was designated as postnatal day (PND) 0. Apart from cage cleaning once a week, dams and their offspring were kept undisturbed until weaning (on PND 25). At weaning, male and female mice were separated and located in same-sex same-litter cages; additionally, male mice were marked through ear clipping and the ear tissue removed through this procedure was used for genotyping. Homozygous knock-out (KO) and WT mice were then used for the experiments.

### Fostering Procedures and Rearing

Fourteen wild type (WT) and 14 St6Gal1 Homozygous (hereafter KO) female mice have been mated with seven WT and seven KO male mice respectively. Out of this batch, 10 WT and 10 KO dams gave birth to a viable offspring. Day of birth has been designated as postnatal day (PND) 0. The fostering procedure (see FIG. 1 for details), performed between 10:00 and 13:00, required the use of four dams (two WT and two KO) at the same time. Thus, to minimise the number of subjects to be discarded due to the absence of foster dams, fostering procedures were performed between 24 and 60 hours after birth. On the day of fostering, we first removed the dams from their cage and then sexed and marked the offspring through toe tattoo ink puncture. After sexing and marking procedures were completed, pups were moved to the cage housing the foster dam and covered with sawdust. Each offspring was transferred to a foster dam in order to expose all experimental subjects to the same condition. Each dam nurtured a mixed litter composed of WT and KO male and female offspring (1:1 ratio among all variables whenever possible). At eye opening and at adult age, prefrontal cortex and hippocampus brain samples were collected for gene expression analyses.

The following experimental groups were constituted:
WT offspring reared to WT dams (WT to WT)
WT offspring reared to KO dams (WT to KO)
KO offspring reared to WT dams (KO to WT)
KO offspring reared to KO dams (KO to KO)

### Total RNA Extraction and QC

Total RNA is extracted using the Agencourt RNAdvance Tissue Kit (Beckman Coulter): Lysis was done in 450 μL. 400 μL of Lyzate was extracted. Elution volume 50 μL Quantification is performed using Quant It Ribogreen assay (Life Technologies) QC assessment is performed using Standard sensitivity RNA kit on Fragment Analyzer 96 (Agilent).

### Samples Libraries Preparation

Libraries are generated using the QuantSeq 3′ mRNA-Seq Library Prep Kit (FWD) HT for Illumina from Lexogen. It is designed to generate Illumina compatible sequences close to the 3′ end of polyadenylated RNA.

The kit uses total RNA as input, hence no prior poly(A) enrichment or rRNA depletion is needed. Library generation starts with oligodT priming containing the Illumina-specific Read 2 linker sequence. After first strand synthesis the RNA is removed. Second strand synthesis is initiated by random priming and a DNA polymerase. The random primer contains the Illumina-specific Read 1linker sequence. No purification is required between first and second strand synthesis. Second strand synthesis is followed by a magnetic bead-based purification step. Sequences required for cluster generation for sequencing are introduced during library amplification step. Double stranded cDNA are amplified by PCR. During this step individual barcodes indexes are introduced in order to multiplex samples. NGS reads are generated towards the poly(A) tail and directly correspond to the mRNA. See on FIG. 1 for details. Libraries are quantified with Quant it Picogreen (Life Technologies). Size pattern is controlled with the High Sensitivity NGS Fragment Analysis kit on a Fragment Analyzer (Agilent). Libraries are pooled at an equimolar ratio (i.e. an equal quantity of each sample library) and clustered at a concentration of 9 pM on single read (SR) sequencing flow cell (Illumina). Sequencing is performed for 65 cycles on a HiSeq 2500 (Illumina) using the HiSeq SR Cluster Kit v4 cBot, HiSeq SBS Kit V4 50 cycle kit (Sequencing by Synthesis). Primary data quality control is performed during the sequencing run to ensure the optimal flow cell loading (cluster density) and check the quality metrics of the sequencing run (QC30). The optimal number of density of clusters detected by image analysis is between 850 and 1000 K/mm2. Performing a run at optimal cluster density involves finding a balance between under clustering that maintains data of good quality but results in lower data output and over clustering that can lead to poor run performance. The percentage ≥Q30, means the percentage of bases with a quality Phred score of 30 or higher. It is a measure of the quality of the identification of the nucleobases generated. The Phred score is logarithmically linked to error probabilities. A Phred score equal to 30 means a probability of incorrect base assignment of 1 in 1000, so a base call accuracy of 99.9%. Regarding Illumina specification, the score should be a minimum of 80%.

### Data Analyses

Genes with very low counts are unlikely to be differentially expressed between groups. We filtered lowly expressed genes by selecting only genes with at least 5 reads in at least 8 samples. The threshold on the number of samples ensures that the genes will be expressed in the smallest group (which contains 8 samples). The filtering step is done on the CPM values which takes the library size into account. In our case it corresponds to a threshold of 2.427 on the CPM values. We also discarded genes with no annotation. We kept 11523 features with these filtering criteria. We normalized using TMM method.

### Results

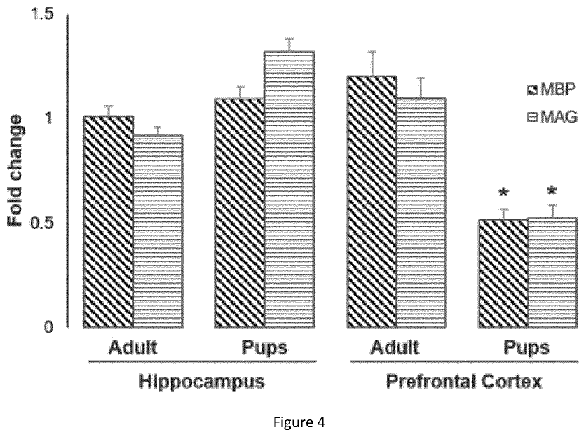

The expression of multiple genes related to myelination was reduced in the mice receiving milk without 6′SL only in the prefrontal cortex during early life, this was neither observed in adulthood nor in the hippocampus at any of the two ages. FIG. 4 show the reduction of 50% of the expression of myelin basic protein (MBP) and myelin-associated glycoprotein (MAG), two key protein of the myelin sheath, in mice receiving milk without 6′SL (gene expression quantified in fold change relative to control mice). When we investigated the impact of absence of 6′SL in early life on the the Kyoto Encyclopedia of Genes and Genomes pathways, we identified that both the myelination and the myelin sheath pathway were down-regulated in mice receiving milk without 6′SL. This again was only visible in the prefrontal cortex during early life, neither in adult prefrontal cortex nor in hippocampus (at adult or early life age). These results clearly indicates that the presence of 6′SL in maternal milk is necessary to observe an optimal myelination of the prefrontal cortex during early life. Interestingly the prefrontal cortex is known to be a key brain region to mediate executive functions.

1. A human milk oligosaccharide or a nutritional composition comprising a human milk oligosaccharide to the subject.

2. Method according to claim 1 wherein, the human milk oligosaccharide is selected from the group consisting of 2′-fucosyllactose (2′FL), diFL, LNT, LNnT, a sialyllactose and any combination of the foregoing.

3. Method according to claim 1 wherein, the subject is a mammal.

4. Method according to claim 3 wherein the subject is a human infant or child.

5. Method according to claim 1 wherein, said composition is selected from the group consisting of an infant formula, a starter infant formula, a follow-on formula, a preterm infant formula, a fortifier, a human milk fortifier, a baby food formula, a growing-up milk, an infant cereal composition, a food product, a medical food product for clinical nutrition, a supplement, a pet food product, and supplement for pets.

6. Method according to claim 1 wherein the human milk oligosaccharide is a combination of 2′fl, diFL, LNT and LNnT, or a combination of 3′-sialyllactose (3′-SL) and 6′-sialyllactose (6′-SL) or a combination of 2′FL, diFL, LNT and LNnT, 3′-sialyllactose (3′-SL) and 6′-sialyllactose (6′-SL).

7. Method according to claim 1, wherein the human milk oligosaccharide is a sialyllactose comprising 3′-SL, 6′-SL or a combination thereof in a human child or infant, for example a pre-term or SGA infant.

8-14. (canceled)

15. A method of preventing and/or treating sub-optimal executive function in a subject, said method comprising the step of administering to said subject a human milk oligosaccharide and/or composition comprising a human milk oligosaccharide.

16. A method of enhancing executive function in a subject, said method comprising the step of administering to said subject a human milk oligosaccharide or composition comprising a human milk oligosaccharide wherein, said human milk oligosaccharide is selected from the group consisting of 2′-fucosyllactose (2′FL), diFL, LNT, LNnT, a sialylllactose and any combination of the foregoing.
